(19) United States
(12) Patent Application Publication
(10) Pub. No.: US 2024/0407809 A1
(43) Pub. Date: Dec. 12, 2024

(54) UNICORTICAL BONE FIXATION AND SURGICAL METHODS

(71) Applicant: University of Utah Research Foundation, Salt Lake City, UT (US)

(72) Inventors: Joseph FEATHERALL, Salt Lake City, UT (US); Joel TURTLE, Salt Lake City, UT (US); Colin S. GREGERSEN, Salt Lake City, UT (US)

(73) Assignee: University of Utah Research Foundation, Salt Lake City, UT (US)

(21) Appl. No.: 18/739,098

(22) Filed: Jun. 10, 2024

Related U.S. Application Data
(60) Provisional application No. 63/472,060, filed on Jun. 9, 2023.

(51) Int. Cl. A61B 17/70; A61B 17/84

(52) U.S. Cl. CPC A61B 17/7053; A61B 17/844

(57) ABSTRACT

A bone anchor may include a shaft having a distal end and a distal toggle element rotatably coupled to the distal end such that the distal toggle element may be rotatable between: a stowed orientation in which the distal toggle element may be insertable into a bone through an aperture formed in a cortex of the bone; and a deployed orientation in which the distal toggle element may be positioned to abut an interior surface of the cortex around the aperture. The bone anchor may also include a flange having a shoulder that may be moveable distally toward the distal toggle element to abut an exterior surface of the cortex.

### CROSS-REFERENCE TO RELATED APPLICATIONS

The present disclosure claims the benefit of U.S. Provisional Patent Application Ser. No. 63/472,060, filed on Jun. 9, 2023 and entitled UNICORTICAL BONE FIXATION AND SURGICAL METHODS, which is incorporated by reference as though set forth herein in its entirety.

### TECHNICAL FIELD

The present disclosure relates to the field of systems, methods and devices for bone fixation. The disclosure further relates to the field of systems, methods and devices for the stabilization of spinal elements for fusion.

### BACKGROUND

Treatment of cervical spine pathology often necessitates stabilization of the bony spinal elements to promote fusion. For patients that require posterior fusion, the most common construct includes the use of lateral mass screws within the cervical spine. This form of fixation is the most common option for bony fixation due to their improved safety profile compared to pedicle screws in the cervical spine.

One limitation of lateral mass screw fixation is the known risk of injury to the vertebral artery or nerve root. These injuries occur due to the design and surgical technique of lateral mass screw implantation. The lateral mass is a small volume of bone that sits posterior to these vulnerable structures. To maximize the fixation strength of these screws, surgeons seek to place screws in as long a length of bone as possible. However, if the drill, tap, or screw breach the anterior cortex of the lateral mass, the vertebral artery and/or the adjacent nerve root are at risk of injury.

Another limitation of current surgical techniques is the suboptimal fixation into osteopenic bone. Whereas pedicle screws in the lumbar and thoracic spine engage a tube of strong cortical bone for optimal fixation, lateral mass screws rely on a very small area of cortical bone in combination with highly variable cancellous bone within the lateral mass. Thus, some patients experience failure of their construct and require revision surgery.

### SUMMARY

The various systems and methods of the present disclosure have been developed in response to the present state of the art, and in particular, in response to the problems and needs in the art that have not yet been fully solved by currently available glenoid replacement systems and methods.

In some embodiments, a bone anchor may include a shaft having a distal end and a distal toggle element rotatably coupled to the distal end such that the distal toggle element may be rotatable between: a stowed orientation in which the distal toggle element may be insertable into a bone through an aperture formed in a cortex of the bone; and a deployed orientation in which the distal toggle element may be positioned to abut an interior surface of the cortex around the aperture. The bone anchor may also include a flange having a shoulder that may be moveable distally toward the distal toggle element to abut an exterior surface of the cortex.

In the bone anchor of any preceding paragraph, the flange may be configured to threadably receive the shaft.

In the bone anchor of any preceding paragraph, the bone anchor may further include a poly-axial tulip configured to be polyaxially adjustably coupled to a proximal end of the shaft.

In the bone anchor of any preceding paragraph, the shaft may include a non-circular cannulation.

In the bone anchor of any preceding paragraph, the distal toggle element may include a disengagement feature configured to be actuated to rotate the distal toggle element from the deployed orientation to the stowed orientation.

In the bone anchor of any preceding paragraph, the flange may further include a drive feature configured to receive torque from a drive instrument to move the shoulder toward the distal toggle element.

In some embodiments a method for securing a bone anchor to a bone may include creating an aperture in a cortex of the bone and positioning the bone anchor proximate the aperture. The bone anchor may include a shaft comprising a distal end, a distal toggle element, and a flange comprising a shoulder. The method may further include, with the distal toggle element in a stowed orientation, advancing the distal toggle element through the aperture, rotating the distal toggle element from the stowed orientation to the deployed orientation, and moving the shoulder distally toward the distal toggle element to abut an exterior surface of the cortex, thereby causing the distal toggle element to abut an interior surface of the cortex around the aperture.

In the method of any preceding paragraph, the flange may be configured to threadably receive the shaft. Moving the shoulder distally toward the distal toggle element may include rotating the flange about the shaft.

In the method of any preceding paragraph, the shaft may include a non-circular cannulation.

In the method of any preceding paragraph, rotating the distal toggle element from the stowed orientation to the deployed orientation may include engaging an actuator with the non-circular cannulation and advancing the actuator distally through the non-circular cannulation to abut the distal toggle element.

In some embodiments, a bone anchor system may include a bone anchor having a shaft comprising a distal end and a distal toggle element rotatably coupled to the distal end such that the distal toggle element may be rotatable between: a stowed orientation in which the distal toggle element may be insertable into a bone through an aperture formed in a cortex of the bone and a deployed orientation in which the distal toggle element may be positioned to abut an interior surface of the cortex around the aperture. The bone anchor system may also include a first actuator configured to rotate the distal toggle element from the stowed orientation to the deployed orientation and a second actuator configured to rotate the distal toggle element from the deployed orientation to the stowed orientation.

In the bone anchor system of any preceding paragraph, the distal toggle element may include a disengagement aperture.

In the bone anchor system of any preceding paragraph, the second actuator mat include a suture configured to pass through the disengagement aperture such that tension on the suture urges the distal toggle element to rotate from the deployed orientation to the stowed orientation.

In the bone anchor system of any preceding paragraph, the shaft may include a non-circular cannulation.

In the bone anchor system of any preceding paragraph, the first actuator may be configured to be slidably received within the non-circular cannulation.

In the bone anchor system of any preceding paragraph, the first actuator may include a counter-torque feature of an inserter.

In some embodiments a method for engaging a bone anchor with a bone may include creating an aperture in a cortex of the bone and positioning the bone anchor proximate the aperture. The bone anchor may include a shaft comprising a distal end, a distal toggle element, and a flange comprising a shoulder. The method may further include advancing the distal toggle clement through the aperture such that the distal toggle element may be within a cancellous portion of the bone and, with the distal toggle element in the cancellous portion, actuating a first actuator to rotate the distal toggle element from the stowed orientation to the deployed orientation.

In the method of any preceding paragraph, the shaft may further include a cannulation and the first actuator may be slidably received in the cannulation. Actuating the first actuator to rotate the distal toggle clement from the stowed orientation to the deployed orientation may include sliding the first actuator through the cannulation such that the first actuator abuts the distal toggle clement to urge the distal toggle element to rotate toward the deployed orientation.

In some embodiments a method for disengaging a bone anchor from a bone, the bone anchor may include a shaft comprising a distal end, and a distal toggle element rotatably coupled to the distal end, may include, with the shaft positioned in an aperture in a cortex of the bone and the distal toggle clement positioned may be a cancellous portion of the bone, actuating an actuator to rotate the distal toggle element from a deployed orientation in which the distal toggle element may be positioned to abut an interior surface of the cortex around the aperture to a stowed orientation. The method may further include, with the distal toggle element in the stowed orientation, withdrawing the distal toggle clement from the bone through the aperture.

In the method of any preceding paragraph, the actuator may include a suture routed through the distal toggle element. Actuating the actuator may include pulling on the suture to pull the distal toggle element toward the stowed orientation.

These and other features and advantages of the present disclosure will become more fully apparent from the following description and appended claims or may be learned by the practice of the implants, systems, and methods set forth hereinafter.

### BRIEF DESCRIPTION OF THE DRAWINGS

Exemplary embodiments of the present disclosure will become more fully apparent from the following description taken in conjunction with the accompanying drawings. Understanding that these drawings depict only exemplary embodiments and are, therefore, not to be considered limiting of the scope of the present disclosure, the exemplary embodiments of the present disclosure will be described with additional specificity and detail through use of the accompanying drawings in which:

FIG. 1A is a perspective view of a bone anchor in a stowed orientation according to an embodiment of the present disclosure.

FIG. 1B is a perspective view of the bone anchor of FIG. 1A in a deployed orientation.

FIG. 2A is a perspective view of a toggle shaft of a bone anchor according to an embodiment of the present disclosure.

FIG. 2B is a front view of the toggle shaft of FIG. 2A.

FIG. 2C is a rear section view of the toggle shaft of FIG. 2A.

FIG. 3A is a perspective view of a toggle shaft of a bone anchor according to an embodiment of the present disclosure.

FIG. 3B is a front view of the toggle shaft of FIG. 3A.

FIG. 3C is a rear section view of the toggle shaft of FIG. 3A.

FIG. 4A is a perspective view of a distal toggle element of a bone anchor according to an embodiment of the present disclosure.

FIG. 4B is a top view of the distal toggle element of FIG. 4A.

FIG. 4C is a side view of the distal toggle element of FIG. 4A.

FIG. 5A is a perspective view of a flange of a bone anchor according to an embodiment of the present disclosure.

FIG. 5B is a front view of the flange of FIG. 5A.

FIG. 5C is a front section view of the flange of FIG. 5A.

FIG. 6A is a perspective view of a flange of a bone anchor according to an embodiment of the present disclosure.

FIG. 6B is a front view of the flange of FIG. 6A.

FIG. 6C is a front section view of the flange of FIG. 6A.

FIG. 7A is a perspective view of a poly-axial head of a bone anchor according to an embodiment of the present disclosure.

FIG. 7B is front view of the poly-axial head of FIG. 7A.

FIG. 7C is a front section view of the poly-axial head of FIG. 7A.

FIG. 8A is a perspective view of a poly-axial capture element of a bone anchor according to an embodiment of the present disclosure.

FIG. 8B is front view of the poly-axial capture element of FIG. 8A.

FIG. 8C is a front section view of the poly-axial capture element of FIG. 8A.

FIG. 9A is a perspective view of a poly-axial tulip of a bone anchor according to an embodiment of the present disclosure.

FIG. 9B is front view of the poly-axial tulip of FIG. 9A.

FIG. 9C is a front section view of the poly-axial tulip of FIG. 9A.

FIG. 10A is a perspective view of a bone anchor in a stowed orientation according to an embodiment of the present disclosure.

FIG. 10B is a front section view and a top section view of the bone anchor of FIG. 10A in a stowed orientation and an exemplary portion of bone.

FIG. 10C is front view of the bone anchor of FIG. 10A in a stowed orientation and an exemplary portion of bone.

FIG. 10D is a front section view of the bone anchor of FIG. 10A in a stowed orientation and an exemplary portion of bone.

FIG. 11A is a perspective view of a bone anchor in a deployed orientation according to an embodiment of the present disclosure.

FIG. 11B is front view of the bone anchor of FIG. 11A in a deployed orientation and an exemplary portion of bone.

FIG. 11C is a front section view of the bone anchor of FIG. 11A in a deployed orientation and an exemplary portion of bone.

FIG. 12A is a perspective view of a bone anchor in a fastened configuration according to an embodiment of the present disclosure.

FIG. 12B is front view of the bone anchor of FIG. 12A in a fastened configuration and an exemplary portion of bone.

FIG. 12C is a front section view of the bone anchor of FIG. 12A in a fastened configuration and an exemplary portion of bone.

FIG. 12D is a perspective view of the bone anchor of FIG. 12A secured to a lateral mass of a vertebra of a cervical spine.

FIG. 12E is a posterior view of the bone anchor of FIG. 12A secured to a lateral mass of the vertebra of the cervical spine.

FIG. 12F is a perspective view of the bone anchor of FIG. 12A secured to a vertebral body of a lumber spine.

FIG. 12G is a side view of the bone anchor of FIG. 12A secured to a vertebral body of the lumber spine.

FIG. 13A is a perspective view of a counter-torque inserter according to an embodiment of the present disclosure.

FIG. 13B is a front view of the counter-torque inserter of FIG. 13A.

FIG. 13C is a front section view of the counter-torque inserter of FIG. 13A engaged with a bone anchor in a stowed orientation according to an embodiment of the present disclosure.

FIG. 13D is a front section view of the counter-torque inserter of FIG. 13A engaged with a bone anchor in a deployed orientation according to an embodiment of the present disclosure.

FIG. 13E is a perspective view of a counter-torque inserter according to an embodiment of the present disclosure.

FIG. 13F is a front section view of the counter-torque inserter of FIG. 13E.

FIG. 13G is a perspective view of a counter-torque inserter according to an embodiment of the present disclosure.

FIG. 14A is a front view of a bone anchor in a deployed orientation including a disengagement actuator according to an embodiment of the present disclosure.

FIG. 14B is a front section view of the bone anchor of FIG. 14A in a deployed orientation including a disengagement actuator.

FIG. 14C is a front view of the bone anchor of FIG. 14A in a stowed orientation including a disengagement actuator.

FIG. 14D is a front section view of the bone anchor of FIG. 14A in a stowed orientation including a disengagement actuator.

FIG. 15A is a perspective view of a bone anchor engagement sub-assembly including a flange, a toggle shaft, and a distal toggle element according to an embodiment of the present disclosure.

FIG. 15B is a side view of the bone anchor sub-assembly of FIG. 15A.

FIG. 15C is a front view of the bone anchor sub-assembly of FIG. 15A.

FIG. 15D is a front section view of the bone anchor sub-assembly of FIG. 15A.

FIG. 16A is a perspective view of a toggle shaft of a bone anchor sub-assembly according to an embodiment of the present disclosure.

FIG. 16B is a front view of the toggle shaft of FIG. 16A.

FIG. 16C is a side view of the toggle shaft of FIG. 16A.

FIG. 17A is a perspective view of a distal toggle element of a bone anchor sub-assembly according to an embodiment of the present disclosure.

FIG. 17B is a top view of the distal toggle element of FIG. 17A.

FIG. 17C is a front view of the distal toggle element of FIG. 17A.

FIG. 17D is a side view of the distal toggle element of FIG. 17A.

FIG. 18A is a perspective view of a flange of a bone anchor sub-assembly according to an embodiment of the present disclosure.

FIG. 18B is a front view of the flange of FIG. 18A.

FIG. 18C is a side view of the flange of FIG. 18A.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged, and designed in a wide variety of different configurations. Thus, the following more detailed description of the embodiments of the devices, systems, and methods, as represented in the drawings, is not intended to limit the scope of the present disclosure but is merely representative of exemplary embodiments of the present disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms are applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means deviation of the distal part of the leg below the knee inward, resulting in a bowlegged appearance. Valgus means deviation of the distal part of the leg below the knee outward, resulting in a knock-kneed appearance.

The present disclosure relates to devices, systems, and methods for unicortical fixation of bone anchors. Those skilled in the art will recognize that the following description is merely illustrative of the principles of the technology, which may be applied in various ways to provide many alternative embodiments. For the purposes of illustrating the concepts of the present design, the present disclosure illustrates bone anchor devices to be used as part of a construct for cervical spinal fusion. However, it will be understood that other variations and uses are contemplated, including, but not limited to, applications in the thoracic spine, the lumbar spine, any bone that includes a cancellous portion, etc.

The present disclosure further illustrates bone anchors configured to be placed into a lateral mass of a cervical spine using a posterior approach. However, the bone anchor may be applied to other bony elements, that include a cortical portion and a cancellous portion, without departing from the scope of the presently disclosed subject matter. Additionally, or alternatively, the bone anchor may be configured for use in a thoracic and/or lumbar spine using a lateral approach in conjunction with a tether system. Additionally, or alternatively, the bone anchor may be configured for use in the lumber and/or thoracic spine using a midline approach to support spinal fusion.

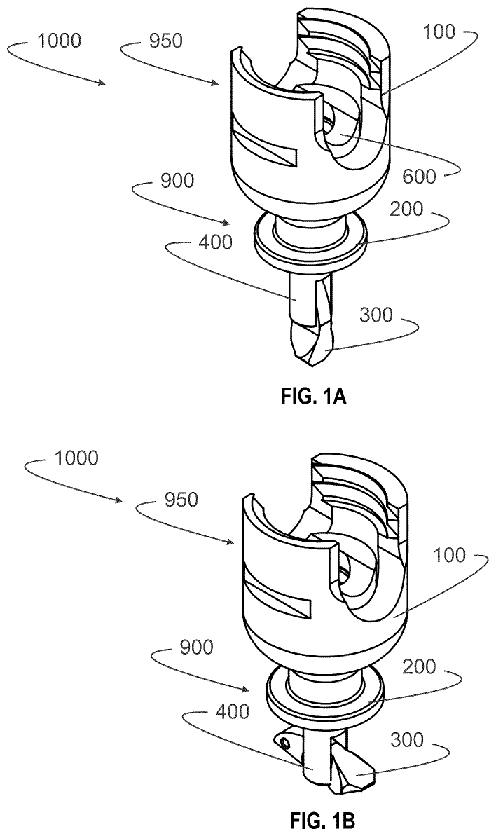

FIG. 1A is a perspective view of a bone anchor 1000 in a stowed orientation according to an embodiment of the present disclosure. FIG. 1B is a perspective view of the bone anchor 1000 in a deployed orientation. The bone anchor 1000 may be configured for unicortical bone fixation and may engage a bone that includes a cortex 1010 and a cancellous portion 1020.

The bone anchor 1000 may include an engagement sub-assembly 900 and a construct portion 950. The engagement sub-assembly 900 may further include a flange 200, a toggle shaft 400, and a distal toggle element 300. In the stowed orientation, the distal toggle element may be insertable into a bone through an aperture 1030 formed in a cortex 1010 of the bone. In the deployed orientation, the distal toggle element may be positioned to abut an interior surface of the cortex 1010 around the aperture 1030.

The flange 200 may be configured to abut an exterior surface of the cortex 1010. The distal toggle element 300 may be configured to rotate between the stowed orientation, in which the distal toggle element 300 may be aligned with a longitudinal axis of the toggle shaft 400, and the deployed orientation, in which the distal toggle element 300 may be generally perpendicular to the longitudinal axis of the toggle shaft 400 and may abut an interior surface of the cortex 1010.

When the flange 200 abuts the exterior surface of the cortex 1010 and the distal toggle element 300 abuts the interior surface of the cortex 1010, the bone anchor 1000 may be secured to the cortex 1010 of the bone. When the bone anchor 1000 is secured to the cortex 1010 of the bone, relative motion between the engagement sub-assembly 900 and the bone may be restricted. However, the construct portion 950 may continue to be moveable relative to the engagement sub-assembly 900. The construct portion 950 may be fixed relative to the engagement sub-assembly 900 when the construct portion 950 is engaged with other implant devices (not shown).

The construct portion may include a poly-axial tulip 100, a poly-axial head 500, and a poly-axial capture element 600. The construct portion 950 may be configured to engage other implant devices (not shown) to form an implant construct. In an embodiment, the construct portion 950 may be configured to receive a spinal rod (not shown) and a fastener (not shown) to secure the rod to the construct portion 950. Additionally, or alternatively, the construct portion 950 may be configured to receive a tether, a plate, a dynamic stabilization system, and/or other implant device configured to be secured relative to a bone.

In an embodiment, the bone anchor 1000 may configured to be used as part of a cervical spinal fusion construct and the proximal engagement portion may be configured as a poly-axial tulip configured to receive a spinal rod (not shown) and a fastener (not shown). In an alternate embodiment, the bone anchor 1000 may configured to be used as part of a vertebral body tethering construct in the thoracic and/or lumber spine.

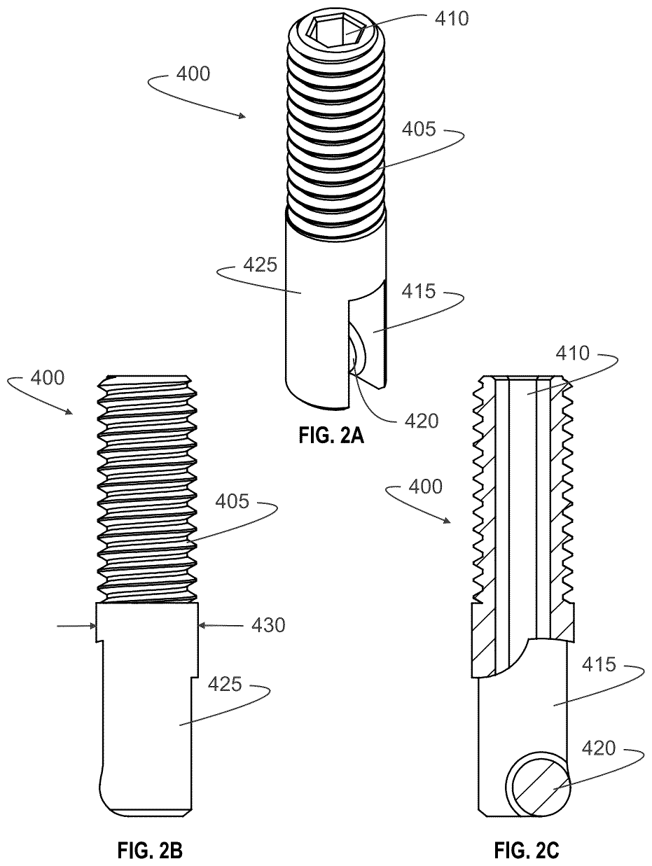

FIG. 2A is a perspective view of a toggle shaft 400 of a bone anchor 1000 according to an embodiment of the present disclosure. FIG. 2B is a front view of the toggle shaft 400 and FIG. 2C is a rear section view of the toggle shaft 400. The toggle shaft 400 may include a threaded portion 405, an internal portion 410, a distal slot 415, and a distal end 425. The toggle shaft 400 may be configured to connect the distal toggle element 300 and the flange 200 and/or the flange 250.

The threaded portion 405 may be configured to threadably engage a threaded portion 210 of a flange 200 and/or a threaded portion 260 of a flange 250. The internal portion 410 may be configured as a non-circular cannulated feature and may be used as a counter-torque feature to restrict rotation of the toggle shaft 400 around a longitudinal axis of the toggle shaft 400 when the flange 200 and/or the flange 250 is threadably rotated about the toggle shaft 400.

The internal portion 410 may be configured as a hexagonal cannulation. Alternatively, the internal portion 410 may be configured as a cannulation with another geometry, such as: a square, a hexalobe, a rectangle, an octagon, an oval, or another non-circular geometry. The internal portion 410 may be configured with a non-circular cannulation compatible with a geometry of a counter-torque portion 715 of a counter-torque inserter 700. The internal portion 410 may be configured to receive the counter-torque portion 715 of the counter-torque inserter 700.

The distal slot 415 may further include a rotation pin 420. The rotation pin 420 may be integral with the toggle shaft 400, alternatively, the rotation pin 420 may be a separate component connected to the toggle shaft 400. A longitudinal axis of the rotation pin 420 may be perpendicular to and offset from the longitudinal axis of the toggle shaft 400. The distal slot 415 may be configured to receive the distal toggle element 300. The rotation pin 420 may be configured to be received in a rotation aperture 320 of the distal toggle element 300. The distal slot 415 and the rotation pin 420 may be configured to allow rotation of the distal toggle element 300 between the stowed orientation and the deployed orientation.

The distal end 425 may include a shaft diameter 430. The distal slot 415 may be included within the distal end 425. The shaft diameter 430 may be generally equal to or less than a major diameter of the threaded portion 405. The distal end 425 may be sized to be received in an inside diameter 225 and the threaded portion 210 of the flange 200 and/or an inside diameter 275 and the threaded portion 260 of the flange 250.

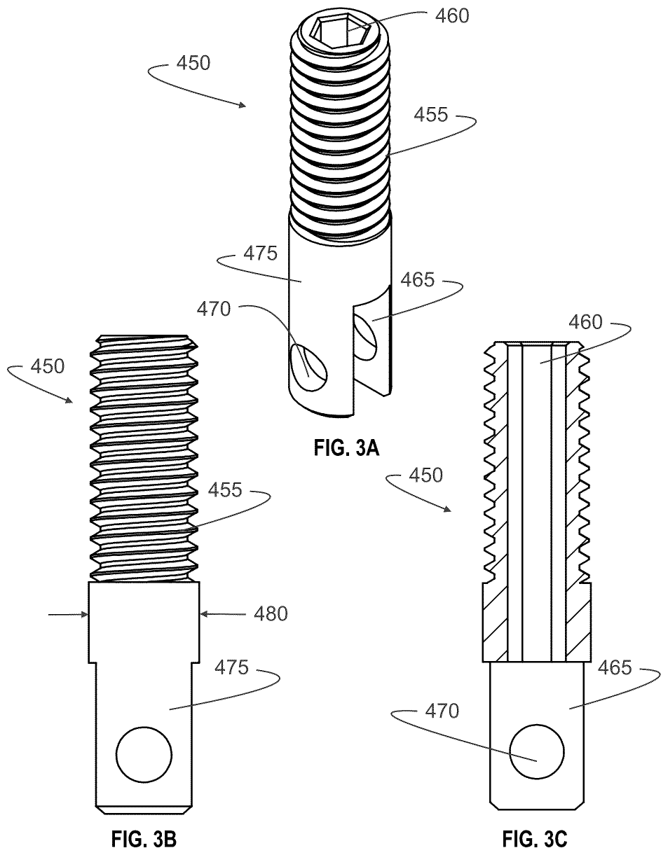

FIG. 3A is a perspective view of a toggle shaft 450 of a bone anchor 1000 according to an embodiment of the present disclosure. FIG. 3B is a front view of the toggle shaft 450 and FIG. 3C is a rear section view of the toggle shaft 450. The toggle shaft 450 may include a threaded portion 455, an internal portion 460, a distal slot 465, and a distal end 475. The toggle shaft 450 may be configured to connect the distal toggle element 300 and the flange 200 and/or the flange 250.

The threaded portion 455 may be configured to threadably engage a threaded portion 210 of a flange 200 and/or a threaded portion 260 of a flange 250. The internal portion 460 may be configured as a non-circular cannulated feature and may be used as a counter-torque feature to restrict rotation of the toggle shaft 450 around a longitudinal axis of the toggle shaft 450 when the flange 200 and/or the flange 250 is threadably rotated about the toggle shaft 450.

The internal portion 460 may be configured as a hexagonal cannulation. Alternatively, the internal portion 460 may be configured as a cannulation with another geometry, such as: a square, a hexalobe, a rectangle, an octagon, an oval, or another non-circular geometry. The internal portion 460 may be configured with a geometry compatible with a geometry of a counter-torque portion 715 of a counter-torque inserter 700. The internal portion 460 may be configured to receive the counter-torque portion 715 of the counter-torque inserter 700.

The distal slot 465 may further include a rotation aperture 470 configured to receive a rotation pin (not shown). The rotation pin (not shown) may be a separate component connected to the toggle shaft 450. An axis of the rotation aperture 470 may be perpendicular to and aligned with the longitudinal axis of the toggle shaft 450. The distal slot 465 may be configured to receive the distal toggle clement 300. The rotation pin (not shown) received in the rotation aperture 470 may be configured to be received in a rotation aperture 320 of the distal toggle element 300. The distal slot 465 and the rotation aperture 470 may be configured to allow rotation of the distal toggle clement 300 between the stowed orientation and the deployed orientation.

The distal end 475 may include a shaft diameter 480. The distal slot 465 may be included within the distal end 475. The shaft diameter 480 may be generally equal to or less than a major diameter of the threaded portion 455. The distal end 475 may be sized to be received in an inside diameter 225 and the threaded portion 210 of the flange 200 and/or an inside diameter 275 and the threaded portion 260 of the flange 250.

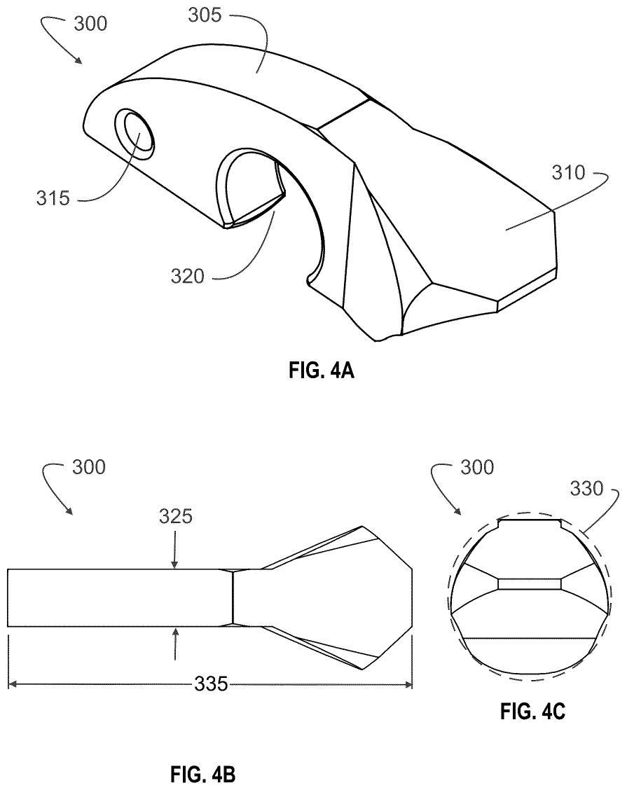

FIG. 4A is a perspective view of a distal toggle clement 300 of a bone anchor 1000 according to an embodiment of the present disclosure. FIG. 4B is a top view of the distal toggle clement 300 and FIG. 4C is a side view of the distal toggle element 300. The distal toggle element 300 may be configured to rotate from a stowed orientation to a deployed orientation. In the stowed orientation, the distal toggle clement 300 may be insertable into a bone through an aperture 1030 formed in a cortex 1010 of the bone. In the deployed orientation, the distal toggle clement 300 may be positioned to abut an interior surface of the cortex 1010 around the aperture 1030.

The distal toggle clement 300 may include an arcuate surface 305, a leading portion 310, and a rotation aperture 320. The rotation aperture 320 may be configured to receive a rotation pin 420 and/or a rotation pin (not shown) that is received within the rotation aperture 470. The distal toggle clement 300 may rotate around an axis of the rotation aperture 320. In the stowed orientation, the leading portion 310 may be directed distally and the arcuate surface 305 may be directed proximally.

Additionally, in the stowed orientation, a portion of the arcuate surface 305 may be aligned with the internal portion 410 of the toggle shaft and/or the internal portion 460 of the toggle shaft 450, so that a counter-torque portion 715 of a counter-torque inserter 700 may travel through the internal portion 410 and/or the internal portion 460 and may abut the arcuate surface 305. The counter-torque inserter 700 may further advance distally through the internal portion 410 and/or the internal portion 460 which may result in the counter-torque portion 715 traveling along the arcuate surface 305 causing the distal toggle element 300 to rotate from the stowed orientation to the deployed orientation.

The distal toggle element 300 may further include a width 325, a length 335, and a profile envelope 330. The width 325 may be configured to allow the distal toggle element 300 to be received within the distal slot 415 and/or the distal slot 465 in the stowed orientation. The length 335 may be configured to be greater than a diameter of an aperture 1030 in a cortex 1010 through which the distal toggle element 300 may pass while in the stowed orientation.

The leading portion 310 may include a profile that is greater than the width 325 which may prevent the leading portion 310 from being received within the distal slot 415 and/or the distal slot 465. The leading portion 310 may include an outside profile that may be bounded by the profile envelope 330. The profile envelope 330 may be less than or generally equal to the shaft diameter 430 and/or the shaft diameter 480. Additionally, the profile envelope 330 may be less than or generally equal to the diameter of the aperture 1030 through which the distal toggle element 300 may pass while in the stowed orientation.

The distal toggle element 300 may further include a disengagement aperture 315. The disengagement aperture 315 may be configured to receive a disengagement actuator 800 and may facilitate rotation of the distal toggle element 300 from the deployed orientation to the stowed orientation.

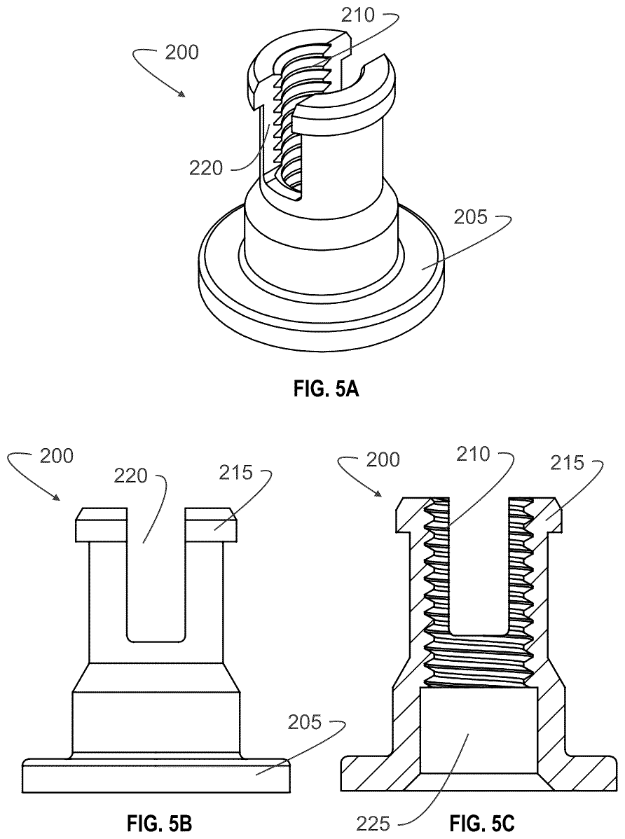

FIG. 5A is a perspective view of a flange 200 of a bone anchor 1000 according to an embodiment of the present disclosure. FIG. 5B is a front view of the flange 200 and FIG. 5C is a front section view of the flange 200. The flange 200 may be configured to threadably receive the toggle shaft 400 and/or the toggle shaft 450. The flange 200 may further be configured to abut an exterior surface of a cortex 1010, and, in conjunction with the distal toggle element 300, secure the bone anchor 1000 to the cortex 1010 of a bone.

The flange 200 may include a shoulder 205, a threaded portion 210, a step portion 215, a slot 220, and an inside diameter 225. The shoulder 205 may be configured to abut an exterior surface of the cortex 1010. The shoulder 205 may be larger than the aperture 1030 through which the distal toggle element 300 may pass while in the stowed orientation. The threaded portion 210 may be configured to threadably receive the threaded portion 405 of the toggle shaft 400 and/or the threaded portion 455 of the toggle shaft 450.

The step portion 215 may be received in an internal step 520 of a poly-axial head 500 thereby retaining the poly-axial head 500 on a proximal end of the flange 200. The slot 220 may allow the step portion 215 to deflect inward during insertion into the internal step 520. The slot 220 may be configured to receive a drive instrument (not shown) wherein the drive instrument may impart torque to the flange 200 to threadably advance the flange 200 along the toggle shaft 400 and/or the toggle shaft 450. The inside diameter 225 may be configured to slidably receive the toggle shaft 400 and/or the toggle shaft 450. The inside diameter 225 may be generally equal to or larger than the major diameter of the threaded portion 210.

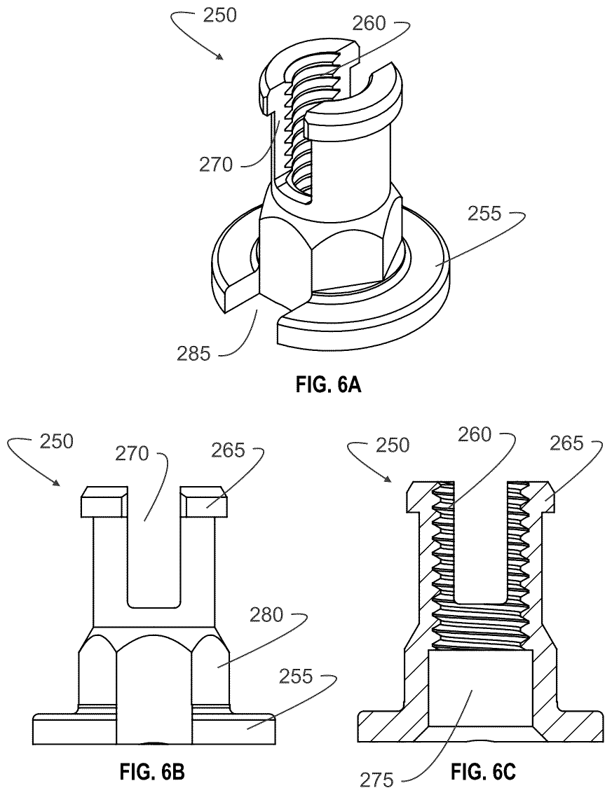

FIG. 6A is a perspective view of a flange 250 of a bone anchor 1000 according to an embodiment of the present disclosure. FIG. 6B is a front view of the flange 250 and FIG. 6C is a front section view of the flange 250. The flange 250 may be configured to threadably receive the toggle shaft 400 and/or the toggle shaft 450. The flange 250 may further be configured to abut an exterior surface of a cortex 1010, and, in conjunction with the distal toggle element 300, secure the bone anchor 1000 to the cortex 1010 of a bone.

The flange 250 may include a shoulder 255, a threaded portion 260, a step portion 265, a slot 270, an inside diameter 275, and a drive feature 280. The shoulder 255 may be configured to abut an exterior surface of the cortex 1010. The shoulder 255 may be larger than the aperture 1030 through which the distal toggle element 300 may pass while in the stowed orientation. The threaded portion 260 may be configured to threadably receive the threaded portion 405 of the toggle shaft 400 and/or the threaded portion 455 of the toggle shaft 450.

The step portion 265 may be received in an internal step 520 of a poly-axial head 500 thereby retaining the poly-axial head 500 on a proximal end of the flange 250. The slot 270 may allow the step portion 265 to deflect inward during insertion into the internal step 520. The inside diameter 275 may be configured to slidably receive the toggle shaft 400 and/or the toggle shaft 450. The inside diameter 275 may be generally equal to or larger than the major diameter of the threaded portion 260. The drive feature 280 may be configured to be received in a drive instrument (not shown) wherein the drive instrument may impart torque to the flange 250 to threadably advance the flange 250 along the toggle shaft 400 and/or the toggle shaft 450. The drive feature 280 may be configured as a hexagon, alternatively other geometries may also be use, such as: a pair of parallel flats, a square, an octagon, or another non-circular geometry.

The shoulder 255 may further include a notch 285. The notch 285 may provide access for a surgeon to drill an additional hole in the cortex 1010 to facilitate removal of a deployed bone anchor 1000.

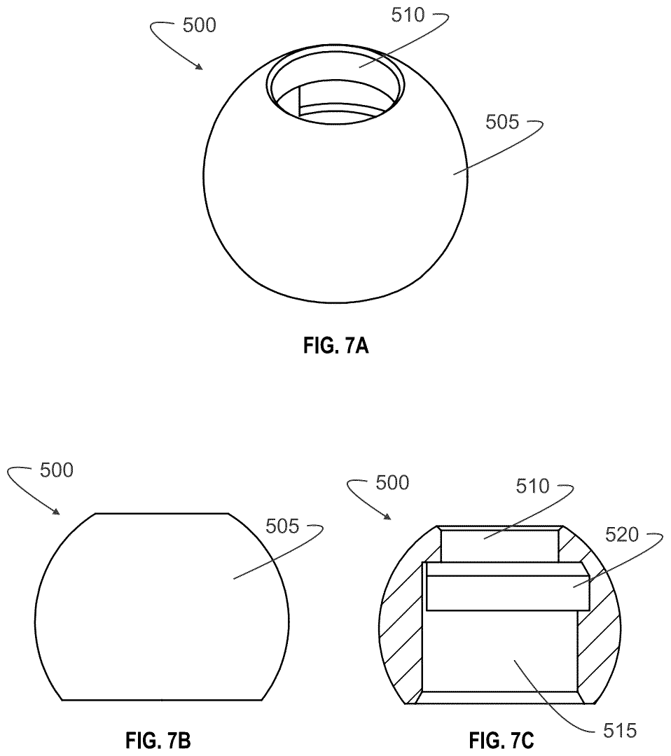

FIG. 7A is a perspective view of a poly-axial head 500 of a bone anchor 1000 according to an embodiment of the present disclosure. FIG. 7B is front view of the poly-axial head 500 and FIG. 7C is a front section view of the poly-axial head 500. The poly-axial head 500 may be configured to facilitate poly-axial movement of a poly-axial tulip 100 relative to the flange 200 and/or the flange 250.

The poly-axial head 500 may include a spherical diameter 505, a first inside diameter 510, a second inside diameter 515, and an internal step 520. The spherical diameter 505 may be received within the poly-axial tulip 100 and may be configured to engage an internal spherical radius 110. The first inside diameter 510 may be configured to allow a counter-torque inserter 700 to pass through the first inside diameter 510 and engage the toggle shaft 400 and/or the toggle shaft 450. The second inside diameter 515 may be configured to receive a proximal end of the flange 200 and/or the flange 250.

The internal step 520 may be configured to receive the step portion 215 of the flange 200 and/or the step portion 265 of the flange 250. Due to the slot 220 of the flange 200, the step portion 215 may encompass less than 360 degrees of circumference. The internal step 520 may also encompass less than 360 degrees of circumference while including sufficient circumference so that the step portion 215 may be fully seated within the internal step 520 without inward deflection of the step portion 215.

Similarly, due to the slot 270 of the flange 250, the step portion 265 may encompass less than 360 degrees of circumference. The internal step 520 may also encompass less than 360 degrees of circumference while including sufficient circumference so that the step portion 265 may be fully seated within the internal step 520 without inward deflection of the step portion 265. Since the internal step 520 may include less than 360 degrees of circumference, rotation of the poly-axial head 500 relative to, and about a longitudinal axis of, the flange 200 and/or the flange 250 may be restricted.

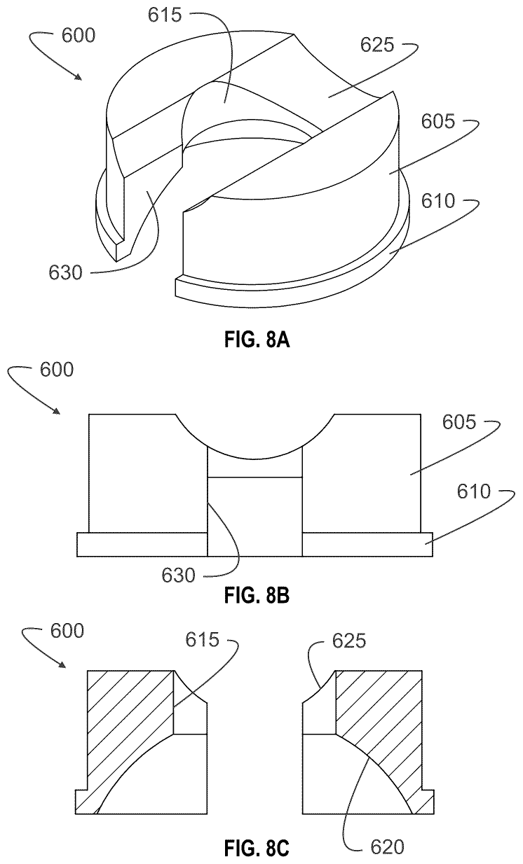

FIG. 8A is a perspective view of a poly-axial capture element 600 of a bone anchor 1000 according to an embodiment of the present disclosure. FIG. 8B is front view of the poly-axial capture element 600 and FIG. 8C is a front section view of the poly-axial capture element 600. The poly-axial capture element 600 may be configured to engage a secondary implant device (not shown) and transfer a force applied by the secondary implant device to the poly-axial head 500, thereby compressing the poly-axial head 500 between the spherical radius 620 of the poly-axial capture clement 600 and the internal spherical radius 110 of the poly-axial tulip, securing the construct portion 950 of the bone anchor 1000 to the engagement sub-assembly 900 of the bone anchor 1000, and restricting poly-axial movement of the construct portion 950.

The poly-axial capture element 600 may include an outside diameter 605, an outside step 610, an inside diameter 615, a spherical radius 620, a rod seat 625 and a slot 630. The outside diameter 605 may be received within an inside diameter 130 of a poly-axial tulip 100. The outside step 610 may be configured to be received within an internal step 105 of the poly-axial tulip 100. When the outside step 610 is received within the internal step 105, the poly-axial capture element 600 may be captive within the poly-axial tulip. The inside diameter 615 may be configured to allow a counter-torque inserter 700 to pass through the inside diameter 615 and engage the toggle shaft 400 and/or the toggle shaft 450.

The slot 630 may extend through the outside diameter 605 and the outside step 610 and into the inside diameter 615. Due to the slot 630, the outside step 610 may encompass less than 360 degrees of circumference. The internal step 105 of the poly-axial tulip 100 may also encompass less than 360 degrees of circumference while including sufficient circumference so that the outside step 610 may be fully seated within the internal step 105 without inward deflection of the outside diameter 605. Since the internal step 105 may include less than 360 degrees of circumference, rotation of the poly-axial capture element 600 relative to, and about a longitudinal axis of, the poly-axial tulip 100 may be restricted. The slot 630 may allow constriction of the outside diameter 605 to facilitate insertion of the poly-axial capture element 600 into the poly-axial tulip 100.

The spherical radius 620 may be configured to engage the spherical diameter 505 of the poly-axial head 500. The rod seat 625 may be configured to receive a secondary implant device (not shown). In an embodiment, the rod seat 625 may be configured to receive a spinal rod (not shown) as part of a spinal fusion construct.

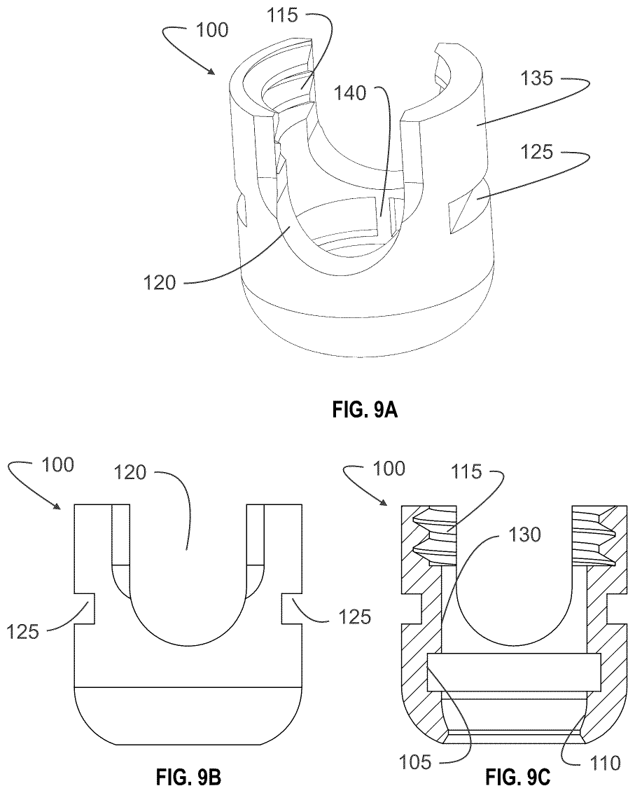

FIG. 9A is a perspective view of a poly-axial tulip 100 of a bone anchor 1000 according to an embodiment of the present disclosure. FIG. 9B is front view of the poly-axial tulip 100 and FIG. 9C is a front section view of the poly-axial tulip 100. The poly-axial tulip 100 may be configured to receive a secondary implant device and a fastener configured to secure the secondary implant device to the poly-axial tulip 100.

The poly-axial tulip 100 may include an internal step 105, an internal spherical radius 110, a threaded portion 115, a rod slot 120, one or more external slots 125, an inside diameter 130, an outside diameter 135, and an alignment feature 140. The rod slot 120 may be configured to receive a secondary implant device. The threaded portion 115 may be configured to receive a fastener (not shown) and allow the fastener to engage the secondary implant device.

The internal step 105 may be configured to receive the outside step 610 of the poly-axial capture element 600. When the outside step 610 is received within the internal step 105, the poly-axial capture clement 600 may be captive within the poly-axial tulip. The inside diameter 130 may be configured to receive the outside diameter 605 of the poly-axial capture element 600. The alignment feature 140 may be located with the internal step 105 and may result in the internal step 105 having less than 360 degrees of circumference. The alignment feature 140 may be received within the slot 630 of the poly-axial capture element 600. The alignment feature 140 may restrict rotation of the poly-axial capture element 600 about a longitudinal axis of the poly-axial tulip 100.

The internal spherical radius 110 may be configured to engage the spherical diameter 505 of the poly-axial head 500. The outside diameter 135 may include one or more external slots 125. The one or more external slots 125 may be used by an external handling device, such as graspers or forceps, for handling and placement of the bone anchor 1000.

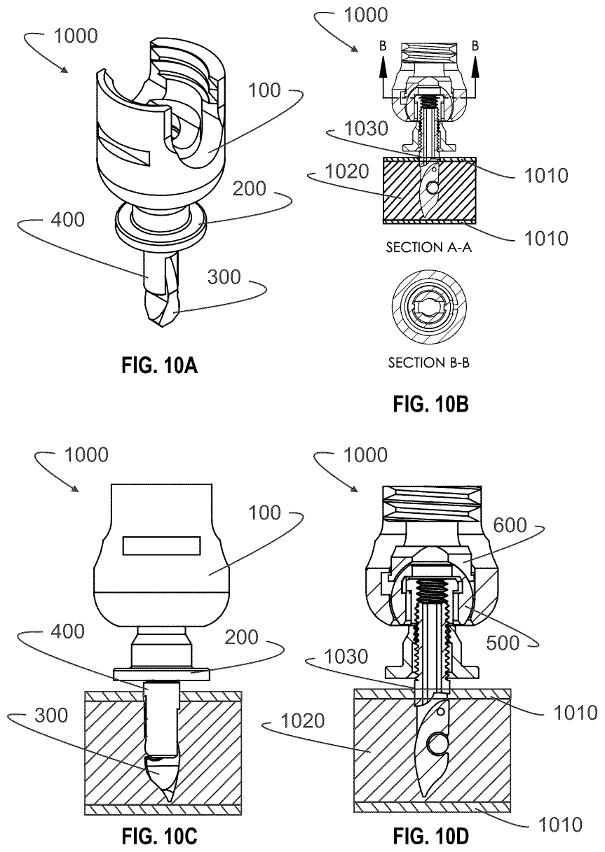

FIG. 10A is a perspective view of a bone anchor 1000 in a stowed orientation according to an embodiment of the present disclosure. FIG. 10B is a front section view and a top section view of the bone anchor 1000 in a stowed orientation and an exemplary portion of bone. FIG. 10C is front view of the bone anchor 1000 in a stowed orientation and an exemplary portion of bone. FIG. 10D is a front section view of the bone anchor 1000 in a stowed orientation and an exemplary portion of bone.

The bone anchor 1000 may include a stowed orientation. In the stowed orientation, the toggle shaft 400 and/or the toggle shaft 450 may be advanced distally with respect to the flange 200 and/or the flange 250. The threaded portion 405 and/or the threaded portion 455 may be threadably engaged with the threaded portion 210 and/or the threaded portion 260 while maximizing the distal travel of the toggle shaft 400 and/or the toggle shaft 450.

Additionally, in the stowed orientation, the distal toggle element 300 may be aligned with a longitudinal axis of the toggle shaft 400 and/or a longitudinal axis of the toggle shaft 450. In the stowed orientation, the distal toggle element 300 and the toggle shaft 400 and/or the toggle shaft 450 may be inserted through an aperture 1030 in a cortex 1010 of a bone. The bone anchor 1000 may be configured so that the distal toggle element 300 may be inserted fully within a cancellous portion 1020 of bone. Additionally, the bone anchor 1000 may be configured so that the distal toggle element 300 may be fully within the cancellous portion 1020 before the flange 200 and/or the flange 250 abuts the exterior of the cortex 1010. The anti-rotation features of the poly-axial tulip 100, the flange 200 and/or the flange 250, poly-axial head 500, and the poly-axial capture element 600 previously described may provide rotational control between the poly-axial tulip and the flange 200 and/or the flange 250.

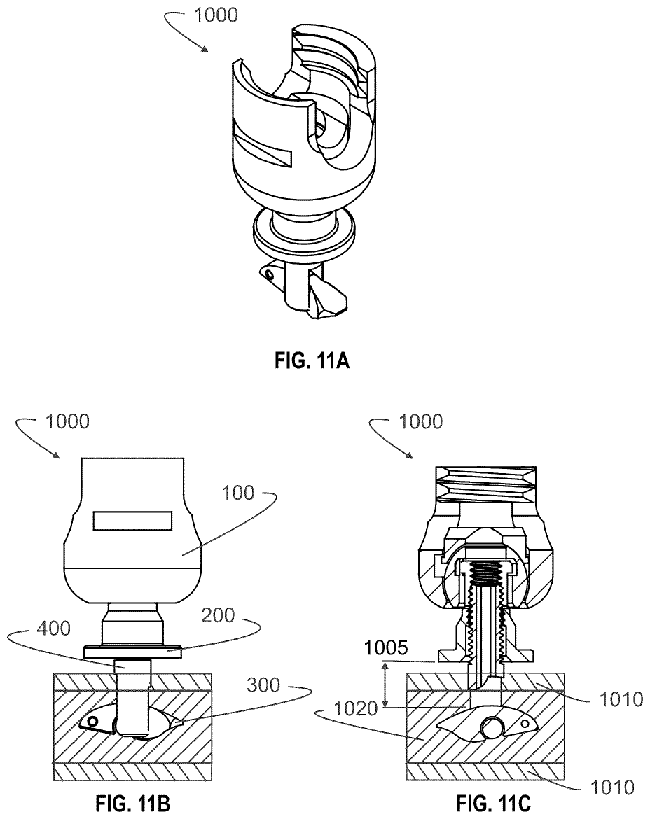

FIG. 11A is a perspective view of a bone anchor 1000 in a deployed orientation according to an embodiment of the present disclosure. FIG. 11B is front view of the bone anchor 1000 in a deployed orientation and an exemplary portion of bone. FIG. 11C is a front section view of the bone anchor 1000 in a deployed orientation and an exemplary portion of bone.

The bone anchor 1000 may also include a deployed orientation. In the deployed orientation, the toggle shaft 400 and/or the toggle shaft 450 may be advanced distally with respect to the flange 200 and/or the flange 250. The threaded portion 405 and/or the threaded portion 455 may be threadably engaged with the threaded portion 210 and/or the threaded portion 260 while maximizing the distal travel of the toggle shaft 400 and/or the toggle shaft 450.

Additionally, in the deployed orientation, the distal toggle element 300 may be generally perpendicular with a longitudinal axis of the toggle shaft 400 and/or a longitudinal axis of the toggle shaft 450. The bone anchor 1000 may be configured so that the distal toggle element 300 may be fully within the cancellous portion 1020 of bone. In the deployed orientation, an engagement distance 1005 may be define as the distance between the distal surface of the shoulder 205 of the flange 200 and/or the shoulder 255 of the flange 250 and the closest surface of the distal toggle element 300. In the deployed orientation, the engagement distance 1005 may be greater than the thickness of the cortex 1010.

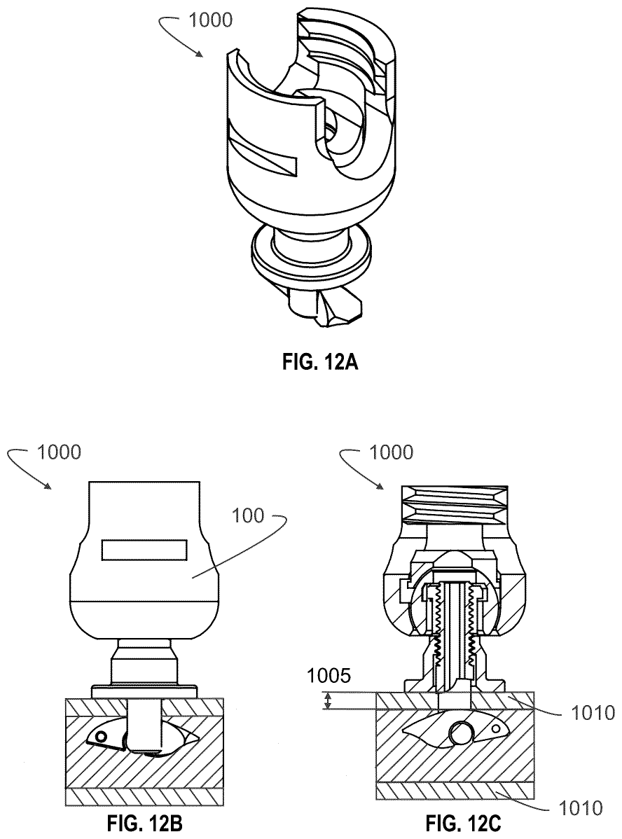

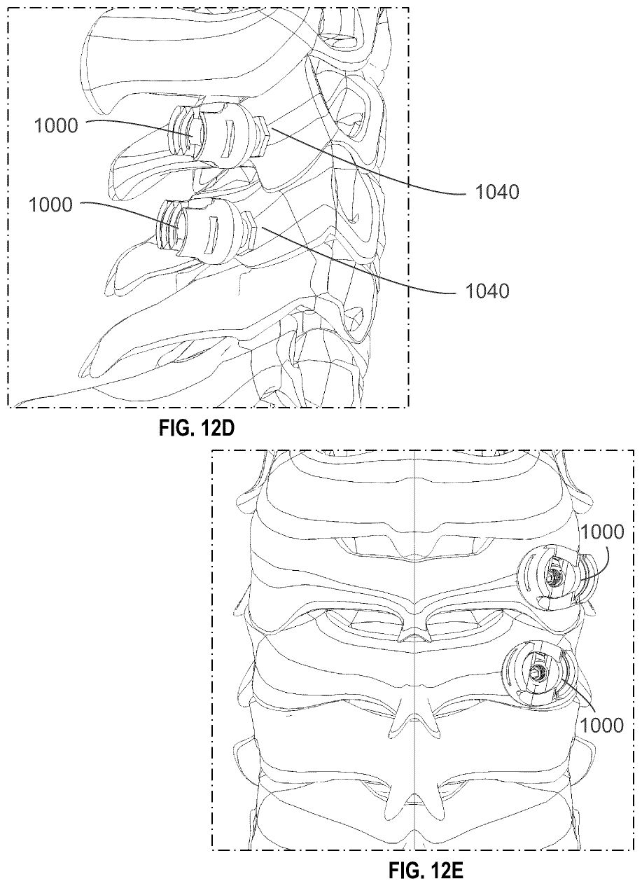

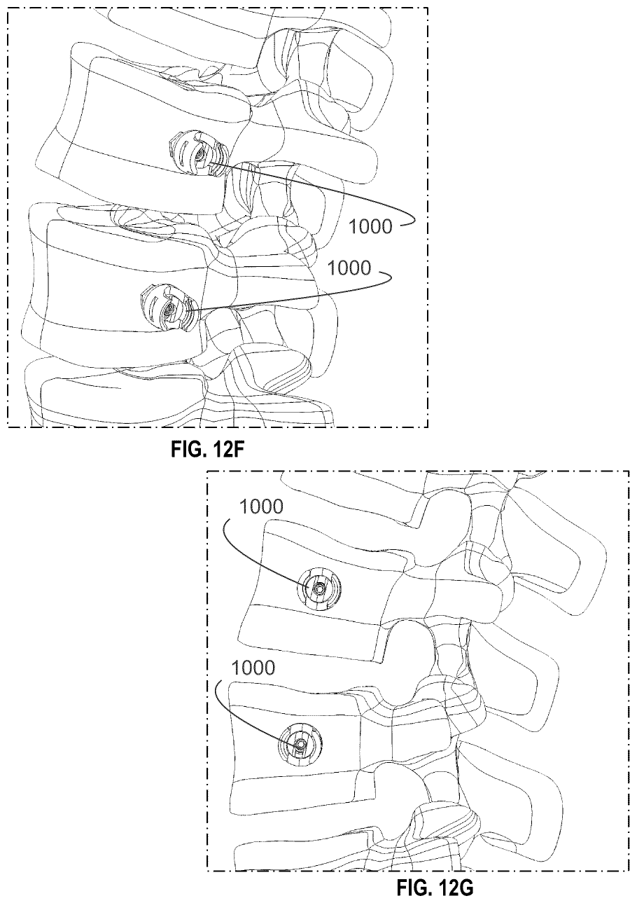

FIG. 12A is a perspective view of a bone anchor 1000 in a fastened configuration according to an embodiment of the present disclosure. FIG. 12B is front view of the bone anchor 1000 in a fastened configuration and an exemplary portion of bone. FIG. 12C is a front section view of the bone anchor 1000 in a fastened configuration and an exemplary portion of bone.

The bone anchor 1000 may also include a fastened orientation. In the fastened orientation, the shoulder 205 of the flange 200 and/or the shoulder 255 of the flange 250 may be moved distally toward the distal toggle element 300 to abut an exterior surface of the cortex 1010, thereby causing the distal toggle element 300 to abut an interior surface of the cortex 1010 around the aperture 1030. The flange 200 and/or the flange 250 may be moved by rotating the flange 200 and/or the flange 250 while inhibiting rotation of the toggle shaft 400 and/or toggle shaft 450, thereby threadably advancing the flange 200 and/or the flange 250 with respect to the toggle shaft 400 and/or the toggle shaft 450.

Additionally, in the fastened orientation, the distal toggle element 300 may be generally perpendicular with a longitudinal axis of the toggle shaft 400 and/or a longitudinal axis of the toggle shaft 450. In the fastened orientation, the engagement distance 1005 may be generally equal to the thickness of the cortex 1010.

FIG. 12D is a perspective view of the bone anchor 1000 secured to a lateral mass 1040 of a vertebra of a cervical spine. FIG. 12E is a posterior view of the bone anchor 1000 secured to a lateral mass 1040 of the vertebra of the cervical spine. The bone anchor 1000 may be configured to be placed into a lateral mass of a cervical spine using a posterior approach. In order to create an implant construct, for example: to facilitate spinal fusion, multiple bone anchors 1000 may be implanted. The multiple bone anchors 1000 may be placed in adjacent vertebrae. Alternatively, the multiple bone anchors 1000 may be implanted so that a vertebra without a bone anchor 1000 may be between two or more vertebrae that have bone anchors 1000 implanted. Additionally, or alternatively, the multiple bone anchors 1000 may be implant on both the medial and the lateral sides of the cervical spine.

FIG. 12F is a perspective view of the bone anchor 1000 secured to a vertebral body of a lumber spine. FIG. 12G is a side view of the bone anchor 1000 secured to a vertebral body of the lumber spine. The bone anchor 1000 may be configured for use in a thoracic and/or lumbar spine using a lateral approach in conjunction with a tether system. In order to create an implant construct, multiple bone anchors 1000 may be implanted.

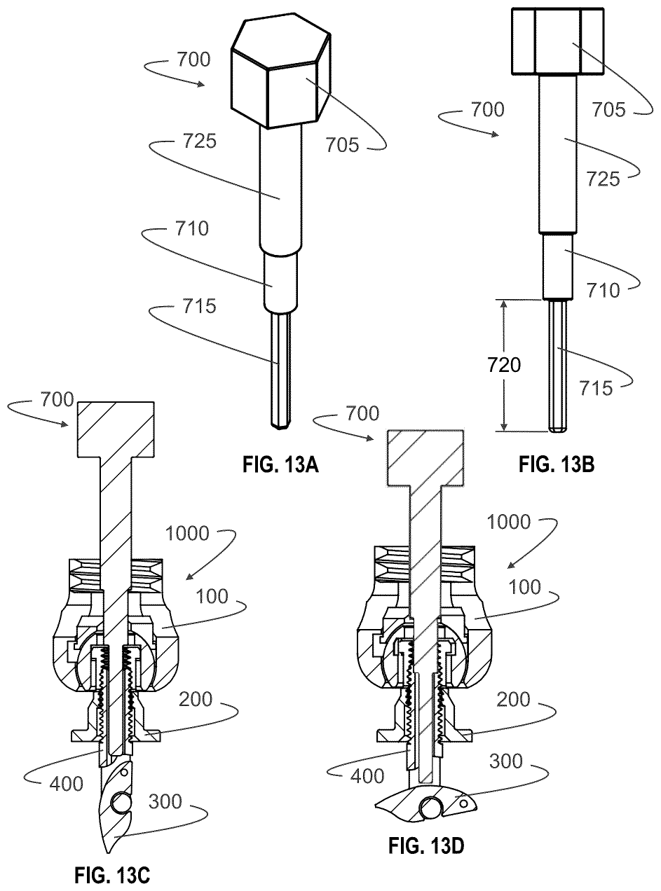

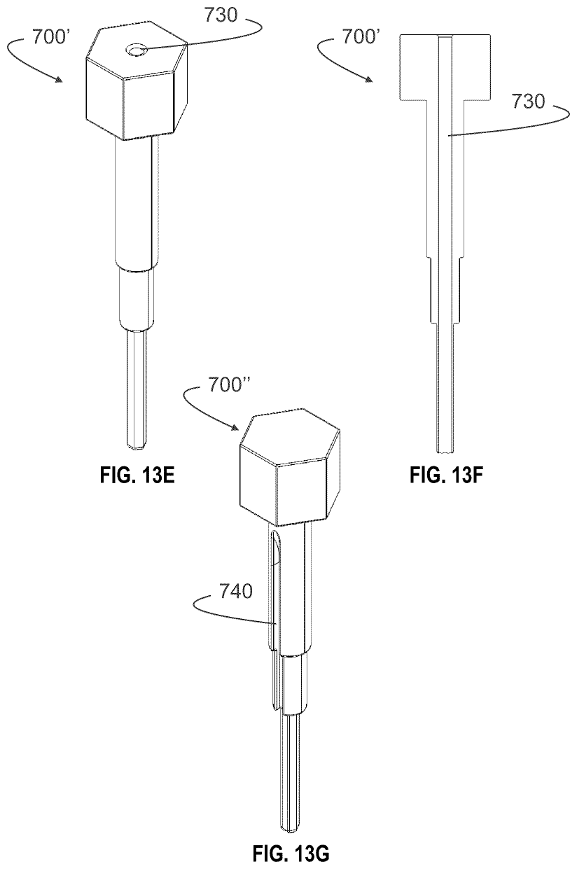

A bone anchor system may also include a counter-torque inserter 700. FIG. 13A is a perspective view of a counter-torque inserter 700 according to an embodiment of the present disclosure. FIG. 13B is a front view of the counter-torque inserter 700. FIG. 13C is a front section view of the counter-torque inserter 700 engaged with a bone anchor in a stowed orientation according to an embodiment of the present disclosure. FIG. 13D is a front section view of the counter-torque inserter 700 engaged with a bone anchor in a deployed orientation according to an embodiment of the present disclosure.

The counter-torque inserter 700 may be configured to inhibit rotation of the toggle shaft 400 and/or the toggle shaft 450 during rotation of the flange 200 and/or the flange 250. Additionally, the counter-torque inserter 700 may be configured to actuate the distal toggle element 300 causing rotation from the stowed orientation and the deployed orientation.

The counter-torque inserter 700 may include a head 705, a first shaft portion 710, a counter-torque portion 715, and a second shaft portion 725. The head 705 may facilitate handling and positioning of the counter-torque inserter 700. Additionally, the head 705 may include a non-circular geometry and/or an indicating feature to provide information to a user as to whether or not the counter-torque inserter 700 has rotated during deployment of the bone anchor 1000.

The second shaft portion 725 may extend distally from the head 705 and the first shaft portion 710 may extend distally from the second shaft portion 725. The first shaft portion 710 may be configured to pass through the inside diameter 615 of the poly-axial capture element 600 and the first inside diameter 510 of the poly-axial head. The second shaft portion 725 may be configured to pass through the inside diameter 130 of the poly-axial tulip 100 and the inside diameter 615 of the poly-axial capture element 600.

The counter-torque portion 715 may extend distally from the first shaft portion 710 and may include a counter-torque length 720. The counter-torque portion 715 may be configured to be received in the internal portion 410 of the toggle shaft 400 and/or the internal portion 460 of the toggle shaft 450. Additionally, the counter-torque portion 715 may include an external geometry that corresponds with the internal geometry of the internal portion 410 of the toggle shaft 400 and/or the internal portion 460 of the toggle shaft 450. The external geometry of the counter-torque portion may be a hexagon, alternatively, the external geometry of the counter-torque portion may be a square, a double-D shape, a hexalobe, or another non-circular geometry.

When the bone anchor 1000 is in the stowed orientation, a portion of the arcuate surface 305 may be aligned with the internal portion 410 of the toggle shaft and/or the internal portion 460 of the toggle shaft 450, so that the counter-torque portion 715 may travel through the internal portion 410 and/or the internal portion 460 and may abut the arcuate surface 305. The counter-torque inserter 700 may further advance distally through the internal portion 410 and/or the internal portion 460 which may result in the counter-torque portion 715 traveling along the arcuate surface 305 causing the distal toggle element 300 to rotate from the stowed orientation to the deployed orientation.

The counter-torque length 720 may be long enough so that the counter-torque portion 715 may contact the distal toggle element 300 when the distal toggle element 300 is in the deployed orientation without the distal travel of the counter-torque inserter 700 being inhibited by the toggle shaft 400 and/or the toggle shaft 450, the poly-axial capture element 600, the poly-axial head 500 or the poly-axial tulip 100.

FIG. 13E is a perspective view of a counter-torque inserter 700′ according to an embodiment of the present disclosure. FIG. 13F is a front section view of the counter-torque inserter 700′. The counter-torque inserter 700′ may include all the features of the counter-torque inserter 700 and may also include a cannulation 730.

FIG. 13G is a perspective view of a counter-torque inserter 700″ according to an embodiment of the present disclosure. The counter-torque inserter 700″ may include all the features of the counter-torque inserter 700 and may also include a groove 740.

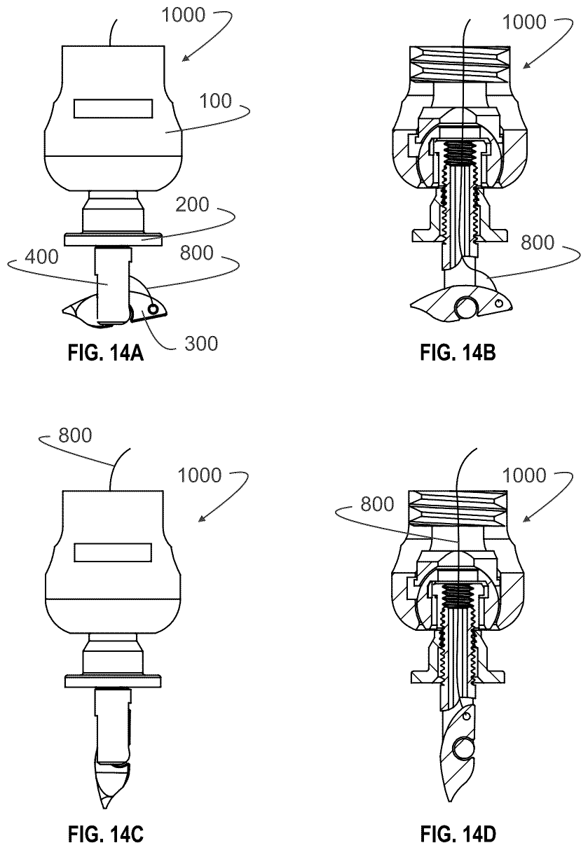

The bone anchor system may further include a disengagement actuator 800. FIG. 14A is a front view of a bone anchor 1000 in a deployed orientation including a disengagement actuator 800 according to an embodiment of the present disclosure. FIG. 14B is a front section view of the bone anchor 1000 in a deployed orientation including a disengagement actuator 800. FIG. 14C is a front view of the bone anchor 1000 in a stowed orientation including a disengagement actuator 800. FIG. 14D is a front section view of the bone anchor 1000 in a stowed orientation including a disengagement actuator 800.

The disengagement actuator 800 may be configured to actuate the distal toggle element 300 from the deployed orientation to the stowed orientation. Actuation of the disengagement actuator 800 may facilitate withdrawal of the bone anchor 1000 from the bone when the bone anchor was previously in the deployed orientation and/or fastened orientation. Withdrawal of the bone anchor 1000 may allow for repositioning of the bone anchor 1000 to a different site on the same bone. Additionally, or alternatively, withdrawal of the bone anchor 1000 may facilitate future revision surgery.

The disengagement actuator 800 may be configured as a length of suture, suture tape, or other flexible thread-like material. The suture material may be monofilament or braided construction. The suture material may be absorbable or non-absorbable. The disengagement actuator 800 may be connected to the disengagement aperture 315 of the distal toggle element 300. The disengagement actuator 800 may be received in the internal portion 410 of the toggle shaft 400 and/or the internal portion 460 of the toggle shaft 450.

The disengagement actuator 800 may further be received in the first inside diameter 510 and the second inside diameter 515 of the poly-axial head 500, the inside diameter 615 of the poly-axial capture element 600, and the inside diameter 130 of the poly-axial tulip 100, thereby allowing the user to grasp the disengagement actuator 800 proximal to the bone anchor 1000 and draw the disengagement actuator 800 proximally to cause rotation of the distal toggle element 300 from the deployed orientation to the stowed orientation.

The counter-torque inserter 700′ may include a cannulation 730 configured to receive the disengagement actuator 800, thereby allowing the counter-torque inserter 700′ to engage the toggle shaft 400 and/or the toggle shaft 450 to inhibit rotation with the disengagement actuator 800 extending proximally from the bone anchor 1000. Additionally, or alternatively, the counter-torque inserter 700″ may include a groove 740 along the length of the counter-torque inserter 700″ configured to receive the disengagement actuator 800, thereby allowing the counter-torque inserter 700″ to engage the toggle shaft 400 and/or the toggle shaft 450 to inhibit rotation with the disengagement actuator 800 extending proximally from the bone anchor 1000.

Additionally, or alternatively, the internal portion 410 of the toggle shaft 400 and/or the internal portion 460 of the toggle shaft 450 may include a groove, along the length of the internal portion, configured to receive the disengagement actuator 800, thereby allowing the counter-torque inserter 700 to engage the toggle shaft 400 and/or the toggle shaft 450 to inhibit rotation with the disengagement actuator 800 extending proximally from the bone anchor 1000.

When the disengagement actuator 800 is received in the counter-torque inserter 700′, the counter-torque inserter 700′ may advance distally through the internal portion 410 and/or the internal portion 460 which may result in the counter-torque portion 715 traveling along the arcuate surface 305 of the distal toggle clement 300 causing the distal toggle element 300 to rotate from the stowed orientation to the deployed orientation with the disengagement actuator 800 extending proximally from the bone anchor 1000.

An exemplary method for securing a bone anchor to a bone may include the following steps:
1. Creating an aperture in a cortex of the bone.
2. Orienting the bone anchor to a stowed orientation.
3. Positioning the bone anchor proximate the aperture.
4. Inserting the distal toggle element through the aperture and into a cancellous portion of the bone.
5. Engaging a counter-torque inserter with the bone anchor.
6. Advancing the counter-torque inserter distally to rotate the distal toggle element from the stowed orientation to a deployed orientation.
5. Using the counter-torque inserter to inhibit rotation of the engagement sub-assembly.
6. Rotating a flange so that a shoulder of the flange moves distally toward the distal toggle element to abut an exterior surface of the cortex.
7. Continue rotating the flange until the distal toggle element abuts an interior surface of the cortex around the aperture, thereby resulting in the bone anchor being in a fastened orientation.

An exemplary method for disengaging a bone anchor from a bone may include the following steps:
1. Optionally engaging a counter-torque inserter with the bone anchor.
2. Optionally using the counter-torque inserter to inhibit rotation of the engagement sub-assembly.
3. Rotating a flange so that a shoulder of the flange moves proximally away from the distal toggle element and an exterior surface of the cortex.
4. Advancing the bone anchor distally so the that distal toggle element no longer abuts an interior surface of the cortex, thereby adjusting the bone anchor from a fastened orientation to a deployed orientation.
5. Actuating a disengagement actuator causing the distal toggle element to rotate from a deployed orientation to a stowed orientation.
6. Withdrawing the distal toggle element from the bone through an aperture.

Those of skill in the art will recognize that this is only one of many potential methods that may be used to secure a bone anchor to a bone and/or disengage a bone anchor from a bone. In alternative embodiments, different methods may be used to secure a bone anchor to a bone and/or disengage a bone anchor from a bone other than the methods described above. Further, the methods set forth above may be used to secure other bone anchors besides those specifically disclosed herein.

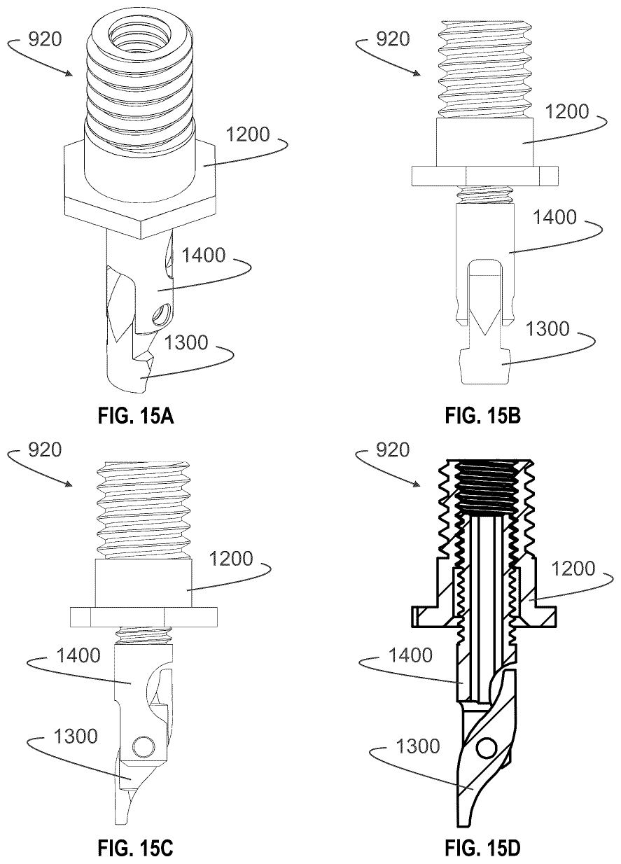

FIG. 15A is a perspective view of a bone anchor sub-assembly 920 including a flange 1200, a toggle shaft 1400, and a distal toggle element 1300 according to an embodiment of the present disclosure. FIG. 15B is a side view of the bone anchor sub-assembly 920, FIG. 15C is a front view of the bone anchor sub-assembly 920, and FIG. 15D is a front section view of the bone anchor sub-assembly 920. The bone anchor sub-assembly 920 may be configured for unicortical bone fixation.

The bone anchor sub-assembly 920 may include a stowed orientation and a deployed orientation. In the stowed orientation, the distal toggle element 1300 may be insertable into a bone through an aperture 1030 formed in a cortex 1010 of the bone. In the deployed orientation, the distal toggle element 1300 may be positioned to abut an interior surface of the cortex 1010 around the aperture 1030.

The bone anchor sub-assembly 920 may be configure to receive a construct portion 950 and/or may be configured to rigidly connect to a secondary implant device. When the bone anchor sub-assembly 920 is secured to the cortex 1010 of the bone, relative motion between the bone anchor sub-assembly 920 and the bone may be restricted.

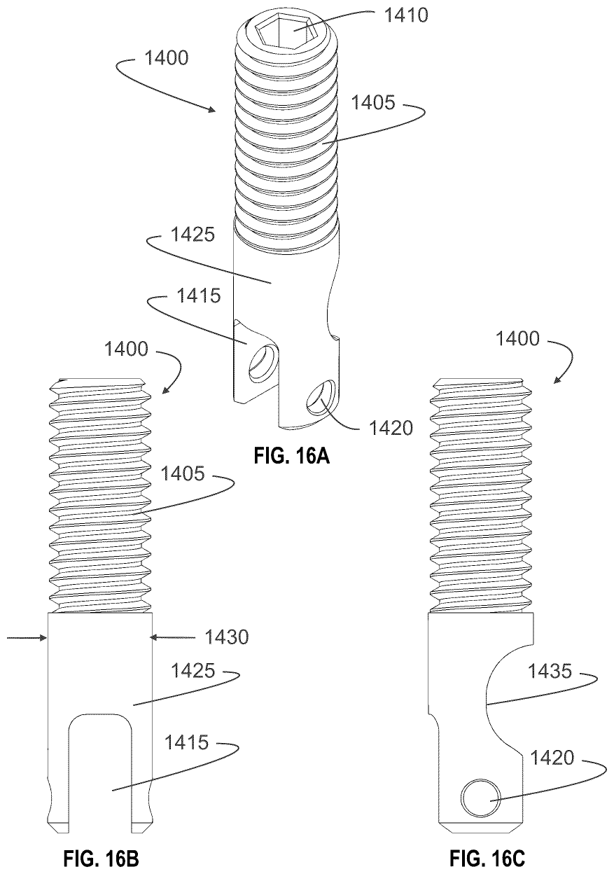

FIG. 16A is a perspective view of a toggle shaft 1400 of a bone anchor sub-assembly 920 according to an embodiment of the present disclosure. FIG. 16B is a front view of the toggle shaft 1400 and FIG. 16C is a side view of the toggle shaft 1400. The toggle shaft 1400 may include a threaded portion 1405, an internal portion 410, a distal slot 415, and a distal end 425. The toggle shaft 1400 may be configured to connect the distal toggle element 1300 and the flange 1200.

The threaded portion 1405 may be configured to threadably engage an inside threaded portion 1260 of a flange 1200. The internal portion 1410 may be configured as a non-circular cannulated feature and may be used as a counter-torque feature to restrict rotation of the toggle shaft 1400 around a longitudinal axis of the toggle shaft 1400 when the flange 1200 is threadably rotated about the toggle shaft 1400.

The internal portion 1410 may be configured as a hexagonal cannulation. Alternatively, the internal portion 1410 may be configured as a cannulation with another geometry, such as: a square, a hexalobe, a rectangle, an octagon, an oval, or another non-circular geometry. The internal portion 1410 may be configured with a geometry compatible with a geometry of a counter-torque portion 715 of a counter-torque inserter 700. The internal portion 1410 may be configured to receive the counter-torque portion 715 of the counter-torque inserter 700.

The distal slot 1415 may further include a rotation aperture 1420 configured to receive a rotation pin (not shown). The rotation pin (not shown) may be a separate component connected to the toggle shaft 1400. An axis of the rotation aperture 1420 may be perpendicular to and aligned with the longitudinal axis of the toggle shaft 1400. The distal slot 1415 may be configured to receive the distal toggle element 1300. The rotation pin (not shown) received in the rotation aperture 1420 may be configured to be received in a rotation aperture 1320 of the distal toggle clement 1300. The distal slot 1415 and the rotation aperture 1420 may be configured to allow rotation of the distal toggle element 1300 between the stowed orientation and the deployed orientation.

The distal end 1425 may include a shaft diameter 1430. The distal slot 1415 may be included within the distal end 1425. The shaft diameter 1430 may be generally equal to or less than a major diameter of the threaded portion 1405. The distal end 1425 may be sized to be received in an inside diameter 1275 and an inside threaded portion 1260 of the flange 1200. The notch 1435 may provide clearance for a trailing portion 1345 of the distal toggle element 1300 when the distal toggle element 1300 is in the stowed orientation.

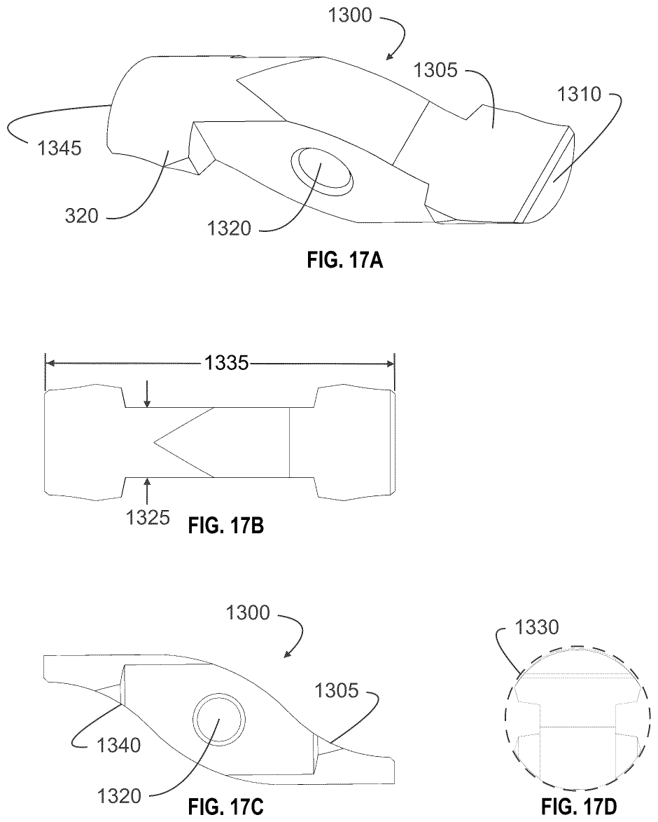

FIG. 17A is a perspective view of a distal toggle element 1300 of a bone anchor sub-assembly 920 according to an embodiment of the present disclosure. FIG. 17B is a top view of the distal toggle element 1300, FIG. 17C is a front view of the distal toggle element 1300, and FIG. 17D is a side view of the distal toggle element 1300. The distal toggle element 1300 may be configured to rotate from a stowed orientation to a deployed orientation. In the stowed orientation, the distal toggle element 1300 may be insertable into a bone through an aperture 1030 formed in a cortex 1010 of the bone. In the deployed orientation, the distal toggle element 1300 may be positioned to abut an interior surface of the cortex 1010 around the aperture 1030.

The distal toggle element 1300 may include a first arcuate surface 1305, a second arcuate surface 1340, a leading portion 1310, a trailing portion 1345, and a rotation aperture 1320. The rotation aperture 1320 may be configured to receive a rotation pin (not shown) that is received within the rotation aperture 1420. The distal toggle element 1300 may rotate around an axis of the rotation aperture 1320. In the stowed orientation, the leading portion 1310 may be directed distally and the first arcuate surface 1305 may be directed proximally.

Additionally, in the stowed orientation, a portion of the first arcuate surface 1305 may be aligned with the internal portion 1410 of the toggle shaft 1400, so that a counter-torque portion 715 of a counter-torque inserter 700 may travel through the internal portion 1410 and may abut the first arcuate surface 1305. The counter-torque inserter 700 may further advance distally through the internal portion 1410 which may result in the counter-torque portion 715 traveling along the first arcuate surface 1305 causing the distal toggle element 1300 to rotate from the stowed orientation to the deployed orientation. Additionally, the leading portion 1310 and the trailing portion 1345 may provide greater surface contact of the distal toggle element 1300 with the interior surface of the cortex as compared to the distal toggle element 300 with the interior surface of the cortex.

The second arcuate surface 1340 and the trailing portion 1345 may be generally the same as the first arcuate surface 1305 and the leading portion 1310 rotated 180 degrees about the rotation aperture 1320. The symmetry of the second arcuate surface 1340 and the trailing portion 1345 with the first arcuate surface 1305 and the leading portion 1310 may provide flexibility related to the orientation during assembly with the toggle shaft 1400.

The distal toggle element 1300 may further include a width 1325, a length 1335, and a profile envelope 1330. The width 1325 may be configured to allow the distal toggle element 1300 to be received within the distal slot 1415 in the stowed orientation. The length 1335 may be configured to be greater than a diameter of the aperture 1030 in a cortex 1010 through which the distal toggle element 1300 may pass while in the stowed orientation.

The leading portion 1310 may include a profile that is greater than the width 1325 which may prevent the leading portion 1310 from being received within the distal slot 1415. The leading portion 1310 may include an outside profile that may be bounded by the profile envelope 1330. The profile envelope 1330 may be less than or generally equal to the shaft diameter 1430. Additionally, the profile envelope 1330 may be less than or generally equal to the diameter of the aperture 1030 through which the distal toggle element 1300 may pass while in the stowed orientation.

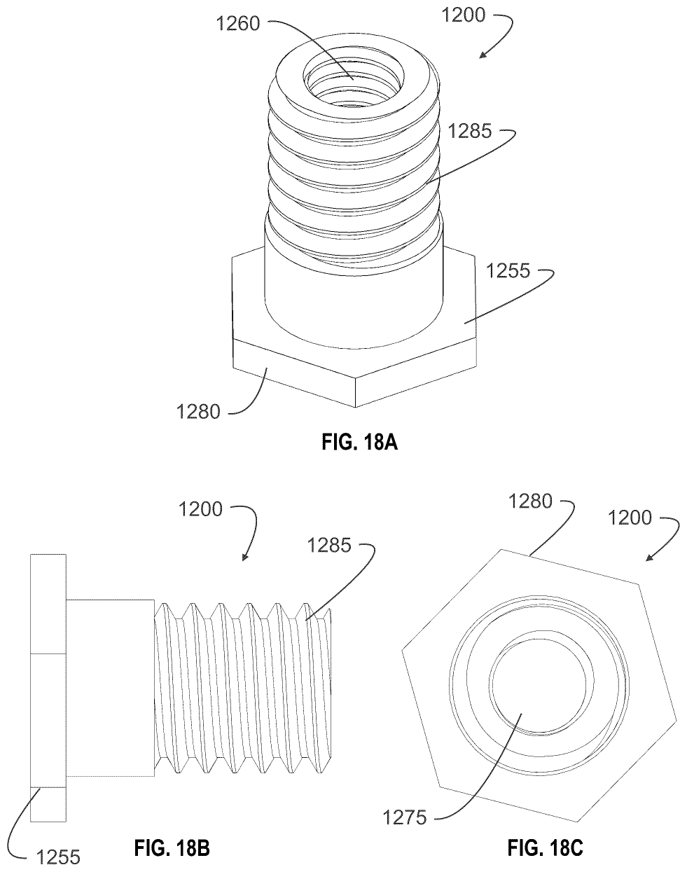

FIG. 18A is a perspective view of a flange 1200 of a bone anchor sub-assembly 920 according to an embodiment of the present disclosure. FIG. 18B is a front view of the flange 1200 and FIG. 18C is a side view of the flange 1200. The flange 1200 may be configured to threadably receive the toggle shaft 1400. The flange 1200 may further be configured to abut an exterior surface of a cortex 1010, and, in conjunction with the distal toggle element 1300, secure the bone anchor sub-assembly 920 to the cortex 1010 of a bone.

The flange 1200 may include a shoulder 1255, an inside threaded portion 1260, an outside threaded portion 1285, and an inside diameter 1275. The shoulder 1255 may be configured to abut an exterior surface of the cortex 1010. The shoulder 1255 may be larger than the aperture 1030 through which the distal toggle element 1300 may pass while in the stowed orientation. The inside threaded portion 1260 may be configured to threadably receive the threaded portion 1405 of the toggle shaft 1400.

The inside diameter 1275 may be configured to slidably receive the toggle shaft 1400. The inside diameter 1275 may be generally equal to or larger than the major diameter of the inside threaded portion 1260. The outside threaded portion 1285 may be configured to securably connect to a secondary implant device (not shown).

The drive feature 1280 may be configured to be received in a drive instrument (not shown) wherein the drive instrument may impart torque to the flange 1200 to threadably advance the flange 1200 along the toggle shaft 1400. The drive feature 1280 may be configured as a hexagon, alternatively other geometries may also be use, such as: a pair of parallel flats, a square, an octagon, or another non-circular geometry.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the present disclosure. This method of disclosure, however, is not to be interpreted as reflecting an intention that any embodiment requires more features than those expressly recited in that embodiment. Rather, inventive aspects lie in a combination of fewer than all features of any single foregoing disclosed embodiment.

Recitation of the term “first” with respect to a feature or element does not necessarily imply the existence of a second or additional such feature or element. Elements recited in means-plus-function format are intended to be construed in accordance with 35 U.S.C. § 112(f). It will be apparent to those having skill in the art that changes may be made to the details of the above-described embodiments without departing from the underlying principles set forth herein.

The phrases “connected to,” “coupled to,” “engaged with,” and “in communication with” refer to any form of interaction between two or more entities, including mechanical, electrical, magnetic, electromagnetic, fluid, and thermal interaction. Two components may be functionally coupled to each other even though they are not in direct contact with each other. The term “coupled” can include components that are coupled to each other via integral formation, as well as components that are removably and/or non-removably coupled with each other. The term “abutting” refers to items that may be in direct physical contact with each other, although the items may not necessarily be attached together. The phrase “fluid communication” refers to two or more features that are connected such that a fluid within one feature is able to pass into another feature. Moreover, as defined herein the term “substantially” means within +/−20% of a target value, measurement, or desired characteristic.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the devices, systems, instruments, and methods disclosed herein.

1. A bone anchor comprising:
a shaft comprising a distal end;
a distal toggle element rotatably coupled to the distal end such that the distal toggle element is rotatable between:
a stowed orientation in which the distal toggle element is insertable into a bone through an aperture formed in a cortex of the bone; and
a deployed orientation in which the distal toggle element is positioned to abut an interior surface of the cortex around the aperture; and
a flange comprising a shoulder that is moveable distally toward the distal toggle element to abut an exterior surface of the cortex.

2. The bone anchor of claim 1, wherein the flange is configured to threadably receive the shaft.

3. The bone anchor of claim 1, further comprising a poly-axial tulip configured to be polyaxially adjustably coupled to a proximal end of the shaft.

4. The bone anchor of claim 1, wherein the shaft comprises a non-circular cannulation.

5. The bone anchor of claim 1, wherein the distal toggle element comprises a disengagement feature configured to be actuated to rotate the distal toggle element from the deployed orientation to the stowed orientation.

6. The bone anchor of claim 1, wherein the flange further comprises a drive feature configured to receive torque from a drive instrument to move the shoulder toward the distal toggle element.

7. A method for securing a bone anchor to a bone, the method comprising:
creating an aperture in a cortex of the bone;
positioning the bone anchor proximate the aperture, the bone anchor comprising:
a shaft comprising a distal end;
a distal toggle element; and
a flange comprising a shoulder;
with the distal toggle element in a stowed orientation, advancing the distal toggle element through the aperture;
rotating the distal toggle element from the stowed orientation to a deployed orientation; and
moving the shoulder distally toward the distal toggle element to abut an exterior surface of the cortex, thereby causing the distal toggle element to abut an interior surface of the cortex around the aperture.

8. The method of claim 7, wherein:
the flange is configured to threadably receive the shaft; and
moving the shoulder distally toward the distal toggle element comprises rotating the flange about the shaft.

9. The method of claim 7, wherein the shaft comprises a non-circular cannulation.

10. The method of claim 9, wherein rotating the distal toggle element from the stowed orientation to the deployed orientation comprises:
engaging an actuator with the non-circular cannulation; and
advancing the actuator distally through the non-circular cannulation to abut the distal toggle element.

11. A bone anchor system comprising:
a bone anchor comprising:
a shaft comprising a distal end; and
a distal toggle element rotatably coupled to the distal end such that the distal toggle element is rotatable between:
a stowed orientation in which the distal toggle element is insertable into a bone through an aperture formed in a cortex of the bone; and
a deployed orientation in which the distal toggle element is positioned to abut an interior surface of the cortex around the aperture;
a first actuator configured to rotate the distal toggle element from the stowed orientation to the deployed orientation; and
a second actuator configured to rotate the distal toggle element from the deployed orientation to the stowed orientation.

12. The bone anchor system of claim 11, wherein the distal toggle element comprises a disengagement aperture.

13. The bone anchor system of claim 12, wherein the second actuator comprises a suture configured to pass through the disengagement aperture such that tension on the suture urges the distal toggle element to rotate from the deployed orientation to the stowed orientation.

14. The bone anchor system of claim 11, wherein the shaft comprises a non-circular cannulation.

15. The bone anchor system of claim 14, wherein the first actuator is configured to be slidably received within the non-circular cannulation.

16. The bone anchor system of claim 11, wherein the first actuator comprises a counter-torque feature of an inserter.

17. A method for engaging a bone anchor with a bone, the method comprising:
creating an aperture in a cortex of the bone;
positioning the bone anchor proximate the aperture, the bone anchor comprising:
a shaft comprising a distal end;
a distal toggle element; and
a flange comprising a shoulder;
advancing the distal toggle element through the aperture such that the distal toggle element is within a cancellous portion of the bone; and
with the distal toggle element in the cancellous portion, actuating a first actuator to rotate the distal toggle element from a stowed orientation to a deployed orientation.

18. The method of claim 17, wherein:
the shaft further comprises a cannulation; and
actuating the first actuator to rotate the distal toggle element from the stowed orientation to the deployed orientation comprises sliding the first actuator through the cannulation such that the first actuator abuts the distal toggle element to urge the distal toggle element to rotate toward the deployed orientation.

19. A method for disengaging a bone anchor from a bone, the bone anchor comprising a shaft comprising a distal end, and a distal toggle element rotatably coupled to the distal end, the method comprising:
with the shaft positioned in an aperture in a cortex of the bone and the distal toggle element positioned in a cancellous portion of the bone, actuating an actuator to rotate the distal toggle element from:
a deployed orientation in which the distal toggle element is positioned to abut an interior surface of the cortex around the aperture, to:
a stowed orientation; and
with the distal toggle element in the stowed orientation, withdrawing the distal toggle element from the bone through the aperture.

20. The method of claim 19, wherein:
the actuator comprises a suture routed through the distal toggle element; and
actuating the actuator comprises pulling on the suture to pull the distal toggle element toward the stowed orientation.
